(19) United States
(12) Patent Application Publication
(10) Pub. No.: US 2019/0133816 A1
(43) Pub. Date: May 9, 2019

(54) ARTICLE COMPRISING A MATERIAL WITH A COOLING FUNCTION

(71) Applicant: ANTARTIC-MEDICAL, La Châtre (FR)

(72) Inventors: Hubert PLANCHON, Châteauneuf-sur-Cher (FR); Olivier PLANCHON, Juvignac (FR)

(73) Assignee: ANTARTIC-MEDICAL, La Châtre (FR)

(21) Appl. No.: 16/096,589

(22) PCT Filed: Apr. 25, 2017
(86) PCT No.: PCT/FR2017/050983

§ 371 Date: Oct. 25, 2018

(30) Foreign Application Priority Data
Apr. 25, 2016 (FR) 1653649

(51) Int. Cl. A61F 7/02; A61F 7/10; A61B 18/02

(52) U.S. Cl. CPC A61F 7/02; A61F 7/10; A61F 2007/0001; A61F 2007/0244; A61F 2007/0271; A61B 18/02

(57) ABSTRACT

Disclosed is an article consisting of at least two sheets stacked and welded to define a paving including at least three tiles, each having at least three sides and vertices, arranged to define a plane, the tiles being adjacent and connected to each other by at least one of the sides, the tiles including at least one compartment including at least a material absorbent to an aqueous medium or a filler material, the tiles being closed and connected to each other by at least one weld. The weld includes a frangible line with a series of weakening welds and junction points or a pre-cut bordered to either side with a mix weld, the mix weld being broadened close to the vertex of each tile so the corner of the tiles is reinforced by an enlarged weld surface area. Also disclosed are a production method and a device.

### FIELD OF THE INVENTION

This invention relates to the field of articles of the thermal compress type. This invention relates in particular to the field of cryotherapy, and in particular that of articles of the refrigerated, deep frozen or frozen thermal compress type.

### PRIOR ART

Cold is well known to be effective in the care of certain physical affections. Cold is useful to relieve pain or to cause the de-swelling of oedema. In certain cases, it is necessary that the cold be applied on the portion of the body to be treated in compliance with conditions of sterility. This is the case for example in the presence of wounds or pronounced irritations of the skin that can be infected. It is moreover known that the analgesic action of the cold is optimal when the skin is brought to a temperature between +5 and +12° C.

Cooling compresses are often pockets that are activated by filling with water then refrigeration or freezing.

American patent application US2008/0027523 describes a thermal blanket for cooling the body of a subject comprising a cooling element comprising a cooling fluid cooled below the freezing point, with this element intended to be placed on the various portions of the body of the subject. In order to obtain a flexible cooling blanket, several cooling elements are advantageously arranged on a flexible support. It can comprise a reinforcement layer formed, for example, by a fabric. However, the cooling elements cannot be detached from one another in order to adjust the dimensions of the cooling blanket to the dimensions or to the morphology of the subject. Thus, this blanket of prior art cannot be adapted in length and in width.

European patent application EP691111 describes a thermal compress comprising a tampon for an application on a member or the body of an animal or of a human, comprising a network of at least two interconnected sealed pockets made from sheets of flexible plastic material, each one of the pockets containing a structured synthetic material that is impregnated with a liquid that has a solidification point less than 0° C. This compress comprises a network of sealed pockets of rectangular shapes linked by welds. However the flexibility of this compress is limited: such a compress, once the pockets are impregnated with the liquid and frozen is finally not very flexible and does not make it possible to hug the curvatures of the zones whereon it is to be applied.

An article is also known from patent EP1607074 comprising at least one outer bag, watertight, comprising on the one hand a compress with a cooling effect and on the other hand a pocket of water, the compress and the pocket of water being in contact with each other. Said compress is formed from an enclosure, at least partially permeable to water, containing particles of a polymer with a high water absorption capacity in the dry state. Said water pocket is closed and assumed to be watertight, but is in direct contact with the enclosure containing the polymer; however, as the material forming the water pocket is notoriously porous, the moisture that escapes therefrom causes premature degradation of the polymer. Therefore, the seal of the water pocket is limited over time. The water pocket comprises a frangible zone of which the rupture makes it possible to transfer water to the enclosure comprising the polymer. The compress can consists of a series of bags aligned according to a single dimension of space. Such a compress, once the pockets impregnated with the liquid and frozen, has the same disadvantages as the other compresses of prior art: it is finally not very flexible. When it is, its flexibility is limited only according to a single dimension of space. Thus, this compress is suitable only for the portions of the body that are substantially flat or cylindrical, such as the abdomen, the arm or the thigh. On the other hand, it cannot hug the curvatures of the zones of the body with complex shapes, such as in particular the hand, the knee, the ankle, the jaw, the cranium, etc.

Thus, the compresses of prior art cannot be adapted in length and in width.

Also known from American patent US2006/0178717 is a thermal blanket that is configurable, made of fabric or of a film that can be torn which avoids pre-ordering a size for a patient. The blanket comprises a plurality of sealed discrete compartments.

Each compartment is entirely circumscribed by a continuous seal that comprises a separation structure in order to allow for the detaching of one or several compartments. However, the risk of tearing the fabric, and therefore the compress, unintentionally when it is sought to separate a compartment, is not managed. Thus, this removable cover of prior art is able to be torn during the detaching of one or several compartments.

Finally, a last need is not covered by the devices of prior art in general and, in particular, those mentioned hereinabove: this is comfort for cold. Indeed, although the application of cold results, in optimum conditions, in the anaesthesia of the skin of the treated zone, it remains that the feeling of cold before the obtaining of this effect can take the form of an unsupportable feeling of burning, which substantially reduces the acceptability of such devices. The feeling of burning is notoriously more pronounced when the cold is applied on a joint, although joints are favoured targets for cryotherapy.

There are therefore multiple needs in the use of articles containing materials with a cooling thermal function, to which the current devices do not respond. In particular, there is a need for an article that is able to:
be adapted in size (length, width and thickness) and in shape,
in certain cases, be able to be packaged in great lengths, for example of several metres, and used progressively according to need,
guarantee the non-tearing during the detaching of a compartment of the article,
deliver the cold in conditions of comfort that are optimal for the patient.

Definitions

In this invention, the terms hereinbelow are defined as follows:
“Adhesive”: material that makes it possible to assemble and to maintain together two objects, reversibly or irreversibly,
“Compartment”: volume closed in the three directions of space, in such a way that this volume does not communicate with the outside, or with other adjacent compartments, unless a wall or a junction has been opened or ruptured,
“Compress”: article of the thermal compress type with a cooling or heating effect,
“Conduction”: method of transporting calories in a material, generally solid, through the progressive propagation of the molecular agitation,
“Convection”: method of transporting calories in a fluid via the movement of the fluid itself,
“Cut”: cut entirely separating two portions of an element,
“Packaging”: packaging intended to contain and to protect merchandise, in particular to prevent the contamination of same, allow for the handling of same and the conveyance of same from the producer to the consumer or to the user, and optionally to ensure the presentation of same,
“Approximately”: the term “approximately”, placed in front of a number, means plus or minus 10% of the nominal value of this number,
“Sheets”: upper and, respectively, lower enclosure of compartments. The two sheets are welded together along lines that delimit said compartments. A sheet can optionally by formed from several stacked thicknesses,
“Reinforcement layer”: designates a fabric or a film stacked on an article in order to guarantee the integrity of same,
“Aqueous medium”: aqueous solution, liquid phase, substantially comprising water, only water, or a mixture of water and dissolved additives; in an embodiment, the aqueous medium can contain an active agent such as described in this invention; in an embodiment, the aqueous medium can contain flavourings or fragrances, colouring agents, active substances that provide a feeling of cold; in an embodiment, the aqueous medium is a saline solution of the physiological serum type,
“Non-woven” manufactured product, formed from a veil, an inlay or fleece distributed directionally or randomly, and of which the internal cohesion is provided by mechanical, physical and/or chemical methods and/or by a combination of these various methods, excluding weaving and knitting,
“Paving”: partition of the plane such that each point of the plane belongs to a tile and to only one. In other terms, a paving comprises tiles that are juxtaposed without overlapping or leaving any space between them. In another embodiment, the tiles can be identical to each other, in that each tile can be deduced from any other tile by symmetry translation, rotation and/or homothety. In another embodiment, the tiles can be of a different form and/or size in the same paving. A tile can have any shape, in particular a tile can be square, rectangular, triangular, hexagonal, circular. A tile can be solid or hollowed. By definition, a paving is infinite. In this invention, it will be said that an article with a finite surface area “is a paving” when it is actually the intersection between a paving strictly speaking and the perimeter of said object,
“Precut”: a zone of the space between two adjacent tiles, over all or a portion of the perimeter of a tile where the two sheets are joined but have a partial cut, generally in the form of a dotted line, making it possible to separate two tiles,
“Absorbent material”: materials or powder that can absorb and retain very large quantities of a liquid compared to its own masse. According to an embodiment, an absorbent material placed in a tile can absorb several times its mass in liquid and thus occupy a volume. The liquid absorbed can be an aqueous medium or an organic liquid,
“Integral”: qualifies the state of two separate elements of the same set that have no relative movement with respect to one another and which are physically connected,
“Mix weld”: assembly of materials that leads to a partial mixture of same and which has for object to provide the continuity of the material to be assembled. The terms mix weld therefore designates here welding in the most conventional acceptance of the term. Welding consists in assembling materials thanks to a method of heating that provokes the partial melting of the parts to be assembled,
“Weakening weld”: weld of materials via mixing of materials and reduction in the thickness of the weld; the reduction in the thickness of the weld has for effect to weaken the weld, which then becomes frangible,
“Enlarged weld”: weld zone along which the weld seam increases in width,
“Junction point”: reinforced connection zone between two tiles, with a width between 0.1 and 2 mm, where the weakening weld is interrupted,
“Frangible line”: alternation of weakening welds and junction points defining a zone via which two tiles can be separated between them,
“In the vicinity”: with respect to a point of a tile located on a length or a width of a tile, means a distance from 1 to 10%, preferably 2 to 5% of the length or of the width of the tile, with respect to this point.

Description

This invention has for object to provide an article that avoids the disadvantages of the devices of prior art, and which responds to the needs mentioned hereinabove.

Thus, the invention relates to, in a first aspect, an article comprised of two sheets stacked and welded to define a paving comprising at least three, preferably at least four tiles; each tile comprises at least three sides and three vertices; these tiles are deployed in at least two directions of space defining a plane; the tiles are adjacent and connected to each other by at least one of the sides of same; the fastening of the perimeter of each tile defines an inside compartment, which is a space between the two sheets, including at least one absorbent material to an aqueous medium or a filler material; the tiles are closed and connected to each other by at least one weld; the weld is carried out via ultrasound, thermally or by any type of welding known to those skilled in the art; the weld consists of a frangible line formed from a succession of weakening welds and junction points or a pre-cut bordered on either side with a mix weld; the mix weld being broadened in the vicinity of each tile such that the corner of the tiles is reinforced by an enlarged weld surface area; preferably, the mix weld has a width in the vicinity of each tile of at least 10% greater than the width of the mix weld of the sides of the tiles; very preferably, the mix weld forms a triangle with sides from 1 to 10, preferably 2 to 8, very preferably 3 to 5 mm at each vertex of the tile.

This broadened weld at the vertices of the tiles makes it possible to reduce the forces that are exerted on the central angle when the compartment is swelled (either by the filler material, or by the absorbent material that was put into contact with an aqueous medium) and through this to prevent tears, in particular during the separation of the tiles.

Moreover, the frangible line is such that it is possible to separate the tiles manually by exerting two forces in the opposite direction and in a direction perpendicular to the plane formed by the tiles. This weld is such, however, that it is not possible to separate the tiles manually by exerting two forces in the opposite direction and in a direction parallel to the plane formed by the tiles.

This invention therefore has for effect to procure a very simple separation of certain tiles from the article, while providing for the integrity of the tiles remaining on the article.

Thus, in an embodiment, the tiles can be detached from one another (before or after activation and/or freezing) without carrying out a tear, without using any particular tool and without weakening the paving structure.

In an embodiment, the weakening weld between two tiles is continuous at the vertex of each tile.

In an embodiment, the weakening weld between two tiles is discontinuous with approximately three points per centimetre, in such a way as to generate at least three junction points.

In a preferred embodiment of the invention, the frangible line is located on the perimeter of the tile, more exactly on all or a portion of the perimeter of the tile.

In an embodiment, the frangible line is located over the entire perimeter of the tile, which makes it possible to detach said tile from the paving wherein it is located.

In an embodiment, the weakening weld is located at the outer edge of a mix weld. In an embodiment, the tile comprises other weakening welds than the weakening weld of the connection with one or several adjacent tiles.

In an embodiment, at least one of the sheets is comprised of a double thickness; preferably, each one of the two sheets is doubled. This doubling has four major advantages. Firstly, it decreases frost during freezing. Then, it substantially increases the comfort to cold. In addition, it delays the migration of the gel contained in the compartment. Finally, it increases the solidity of the article, which allows it to be reused several times.

In an embodiment, the article does not comprise a reinforcement layer.

In a first embodiment, the article is non-sterile.

In a second embodiment, the article is sterile. Preferably, it is sterilised by gamma rays or according to sterilisation methods known to those skilled in the art.

In a third embodiment, the article is decontaminated by gamma rays.

In an embodiment, the surface of the compartment occupies 10 to 99%, preferably 50 to 95%, very preferably 70 to 90% of the surface of the tile.

In a first embodiment, the tile has a shape that is identical to that of the compartment or compartments that it delimits.

In a second embodiment, the tile has a shape that differs from that of the compartment or compartments that it delimits.

In an embodiment, the material absorbent to an aqueous medium comprises or is formed from a chain of poly(acrylic acid) or from the salts thereof. In an embodiment, the material absorbent to an aqueous medium is formed from a single type of polymer.

In an embodiment, the material absorbent to an aqueous medium does not comprise or is not formed from a chain of poly(acrylic acid) or from the salts thereof.

In an embodiment, the material absorbent to an aqueous medium comprises homogeneous particles and does not comprise particles in two parts of the core and shell type.

In an embodiment, the material absorbs 0.1 to 20% of the water capacity that it could absorb in the open air.

According to an embodiment, at least one of the outer faces of the sheets is coated, entirely or partially, with an adhesive.

According to an embodiment, at least one of the outer faces of the sheets is coated, entirely or partially, with a material that adheres without sticking to the skin, for example of the silicone type, in particular a silicone gel.

According to an embodiment, at least one of the sheets is permeable to an activation liquid, which is preferably water or an aqueous medium.

According to an embodiment, at least one sheet, and preferably all of the sheets, are made of a non-woven fabric.

According to an embodiment, the sheets do not include any insulating layer or insulating material.

In an embodiment, the tiles are not thermoformed.

In a particular embodiment of the invention, at least one of the two sheets is thermoformed in order to form, with the other stacked sheet, compartments in three dimensions, in particular parallelepiped, polyhedral, spherical or hemispheric. In an embodiment, the article is surrounded by an edging, which is a longitudinal edge. The edging can be carried out by fastening of the two sheets over all or a portion of the edges of the article.

In an embodiment, the article is in a packaging that comprises at least one microporous or microperforated film permeable to water but a barrier to microbes, bacteria and/or viruses.

In another embodiment, the packaging is multi-pocket and comprises a first pocket comprising the article, a second pocket, independent of the first pocket and sealed with respect to the first pocket, comprising or able to receive an aqueous medium. The two pockets are integral via one of the sides of same and include a means for putting into fluid communication from the second pocket to the first pocket.

In all of the embodiments comprising a packaging, the packaging comprises at least one weld that can be peeled or torn, and preferably at least two adjacent welds that can be peeled, for an easy extraction of the article.

Thus, the invention relates to an article which can be filled with a material absorbent to an aqueous medium and which can be activated by putting into contact with an aqueous medium, said activation having for effect to swell a mixture of water and absorbent material that is inside the inside compartment of the tile. The activation is followed by a step of cooling, freezing or deep freezing. Alternatively, the article according to the invention is filled and swelled with a filler material and requires only being cooled, frozen or deep frozen in order to form a thermal compress.

In a second aspect, the invention therefore relates to a thermal compress comprising an article according to the invention, which was activated if necessary (if the material containing it is a material absorbent to an aqueous medium, the activation is done by putting the article according to the invention in contact with an aqueous medium) and cooled, frozen or deep frozen. In an embodiment, the thermal compress according to the invention has for function to communicate cold to any object or body in contact with same, via transport of the latent melting heat absorbed during the transition from the solid phase to the liquid phase. In an embodiment, the thermal compress according to the invention has for function to communicate cold to any object or body in contact with same, by convection or by conduction.

In a third aspect, the invention relates to a roller formed by the winding on itself of the article according to the invention.

In a fourth aspect, the invention relates to a hat comprising an article according to the invention. More preferably, this hat comprises or is formed from a hairnet, preferably disposable, and from at least one article according to the invention. Advantageously, this hat is a chemotherapy hat intended to combat hair loss linked to a chemotherapy treatment.

In a fifth aspect, the invention relates to a method for manufacturing the article according to the invention comprising a continuous flow of at least two non-welded sheets in front of two welding stations: one longitudinal, another transversal. The longitudinal welding station comprises at least 3 welding zones namely a first edge zone, at least one intermediate zone and a second edge zone, the longitudinal weld being a weakening weld bordered on two mix welds. The transversal welding station comprises at least three welding zones, namely a first edge zone, at least one intermediate zone and a second edge zone, said transversal weld being a weakening weld bordered on two mix welds.

The distance between two longitudinal welding zones defines the length of a tile and the distance between two transversal zones defines the width of a tile.

The method of manufacturing further comprises the depositing of a material absorbent to an aqueous medium in each tile prior to the complete welding of same.

In a sixth aspect, the invention relates to a device for implementing the method according to the invention. The device comprises a longitudinal welding station comprising at least three means of welding, each means comprising 2 mix weld washers surrounding a weakening weld washer that can have notches, in such a way as to create a discontinuous weakening weld generating mix weld junction points, or without a notch, in such a way as to create a continuous weakening weld. The device comprises moreover a transversal welding station comprising 2 mix weld washers surrounding a weakening weld washer that can have notches, in such a way as to create a discontinuous weakening weld, or without a notch, in such a way as to create a continuous weakening weld.

### BRIEF DESCRIPTION OF THE FIGURES

FIGS. 1A, 2A, 3A, 4A and 5A are front views showing several embodiments of a tile according to the invention.

FIGS. 1B, 2B, 3B, 4B and 5B are front views showing several embodiments of the article according to the invention.

FIG. 6 is a cross-section of an embodiment of the article according to the invention.

FIG. 7A is a front view of a thermoformed article according to the invention.

FIG. 7B is a side view of a thermoformed article according to the invention.

FIG. 8 shows an article according to the invention included in a multiple-pocket packaging.

FIG. 9A and 9B show an article according to the invention included in a single-pocket packaging.

FIG. 10 shows the use of an article according to the invention, for cutaneous application on a knee.

FIG. 11 shows a device for manufacturing of an embodiment of the article according to the invention.

### REFERENCES

1—Article,

2—Tile,

3—Compartment,

4—Sheet,

4A—Outer face of the sheet,

4B—Inner face of the sheet,

5—Mix weld,

6—Weakening weld,

7—Junction point,

8—Frangible line,

9—Device for manufacturing,

91—Mechanical parts for guiding and stacking sheets,

92—Vertical fastening module,

93—Horizontal fastening module,

921, 931—Ultrasound unit,

922, 932—Fastening drum,

94—Conveyor belt,

941—Conveyor belt,

942—Motor,

95—Distribution unit,

951—Bag,

952—Transfer cam,

953—Intermediate reservoir,

954—Mechanical batcher,

955—Mechanical corridor,

10—Multi-pocket packaging,

101—First pocket,

102—Second pocket,

11—Single-pocket packaging,

12—Means for putting into fluid communication,

13—Weld that can be peeled or torn.

### DETAILED DESCRIPTION

This invention shall be understood better when reading the description of the figures which show the invention in a non-limiting way.

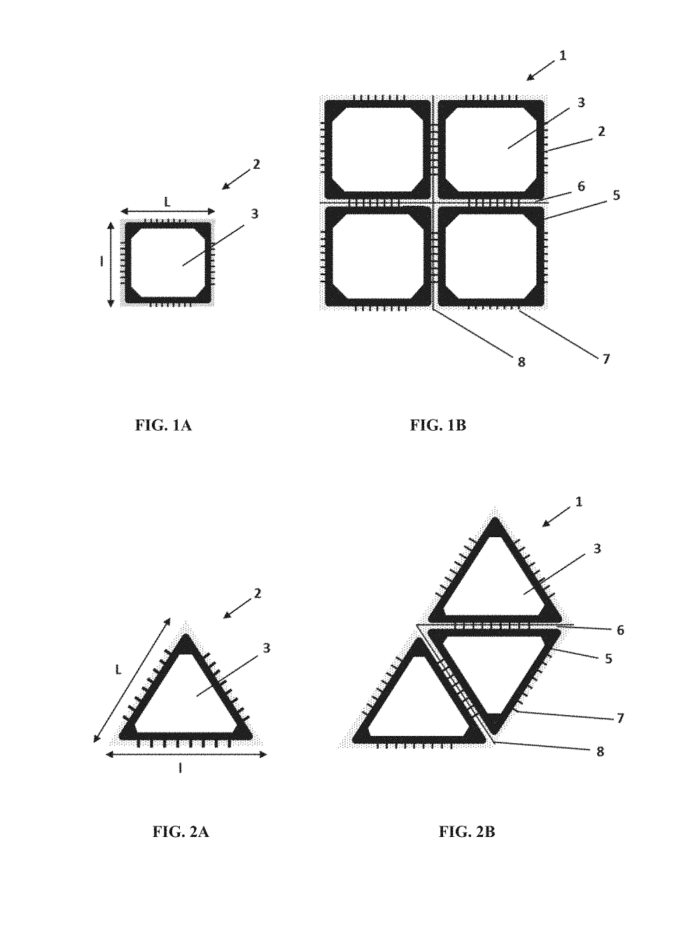

FIGS. 1A, 2A, 3A, 4A and 5A show various embodiments of a tile 2 according to the invention as a front view. Each tile 2 comprises a compartment 3.

In particular FIG. 1A shows a tile 2 of square shape comprising a compartment 3 of square shape; the tile 2 is of dimensions that are slightly greater than those of the compartment 3.

FIG. 2A shows a tile 2 of triangular shape comprising a compartment 3 of triangular shape; the tile 2 is of dimensions that are slightly greater than those of the compartment 3.

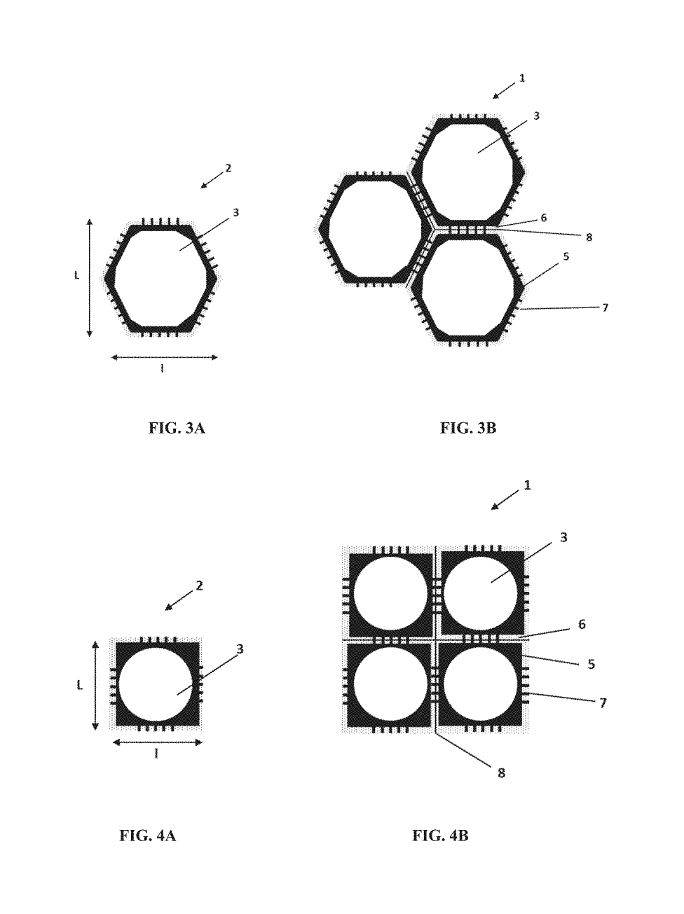

FIG. 3A shows a tile 2 of hexagonal shape comprising a compartment 3 of hexagonal shape; the tile 2 is of dimensions that are slightly greater than those of the compartment 3.

FIG. 4A shows a tile 2 of square shape comprising a compartment 3 of circular shape; the tile 2 is of dimensions that are slightly greater than those of the compartment 3.

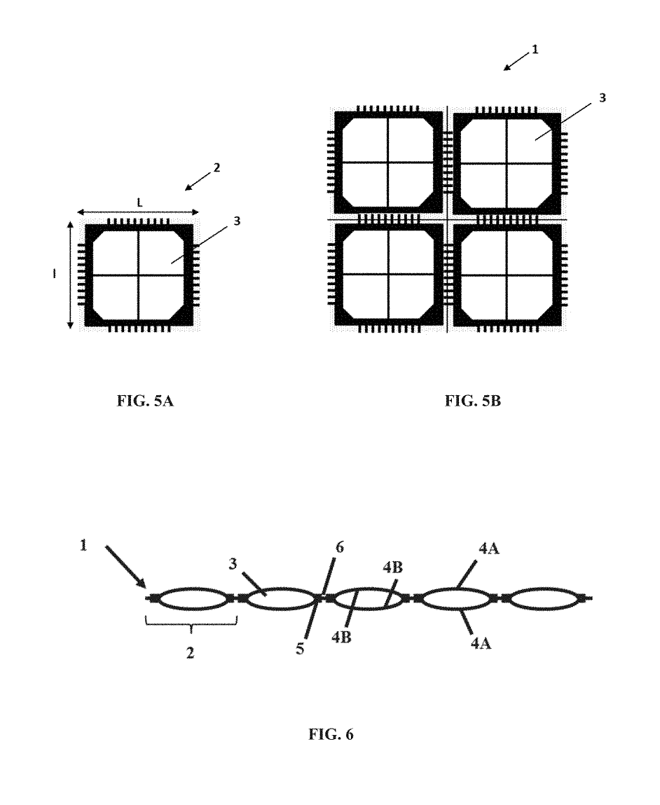

FIG. 5A shows a tile 2 of square shape comprising four compartments 3 of square shape; the tile 2 is of dimensions that are slightly greater than those of the four compartments 3.

FIGS. 1B, 2B, 3B, 4B and 5B show various embodiments of an article 1 view from the front comprising a plurality of tiles 2 such as shown respectively in FIGS. 1A, 2A, 3A, 4A and 5A.

The article 1 comprises at least three tiles 2. This property provides it with the functionality, sought by the invention, of being flexible in all directions.

The article 1 comprises two sheets 4 which each have an outer face 4A and an inner face 4B.

As shown in FIG. 6, each tile 2 comprises at least one compartment 3, delimited by two mix welds (5) that bordure a weakening weld (6). These welds fasten the inner faces 4B of the sheets. The tiles 2 are connected to each other via a weakening weld along the perimeter of same; this weakening weld makes the tiles 2 able to be separated with bare hands, without requiring any tool.

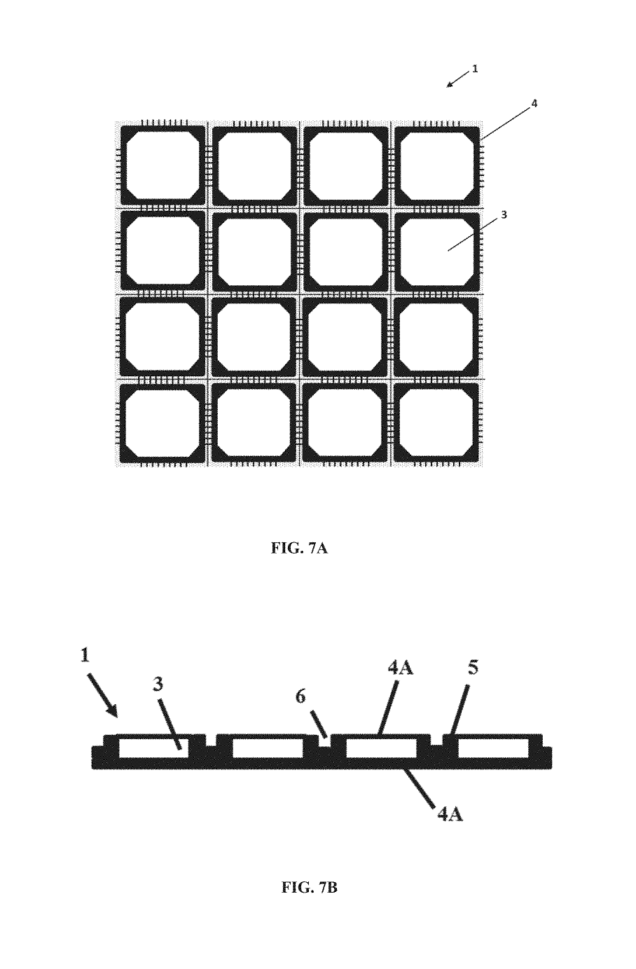

FIGS. 7A and 7B have for object to show respectively a front view and a side view of a particular embodiment of the invention, wherein at least one of the two sheets 4 is thermoformed in order to form, with the other stacked sheet 4, compartments 3 in three dimensions, in particular parallelepiped, polyhedral, spherical or hemispheric. In particular, in FIG. 7B, the upper sheet 4 is thermoformed and the lower sheet 4 is not thermoformed.

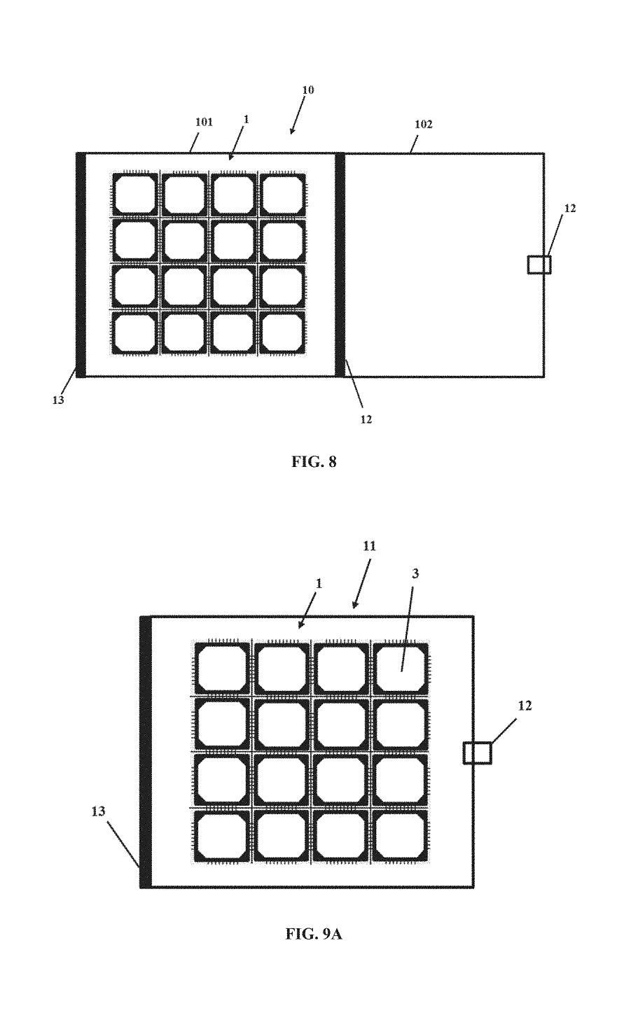

FIG. 8 shows an article 1 according to the invention, which is included in a multiple-pocket packaging 10. Advantageously, the multiple-pocket packaging 10 comprises:
a first pocket 101 comprising an article 1 according to the invention,
a second pocket 102, comprising (or able to comprise) an activation liquid that is sterile or not, of the material with a thermal function,
the two pockets 101, 102 are juxtaposed via one of the sides of same and are preferably integral, on said side; the pocket 101 has a weld that can be peeled or torn 13 making it possible to extract the article; in an embodiment, the pocket 101 comprises at least two adjacent welds 13 that can be peeled,
the two pockets comprising a means for putting into fluid communication 12, which is preferably a weld that can be peeled, between the second pocket 102 and the first pocket 101.

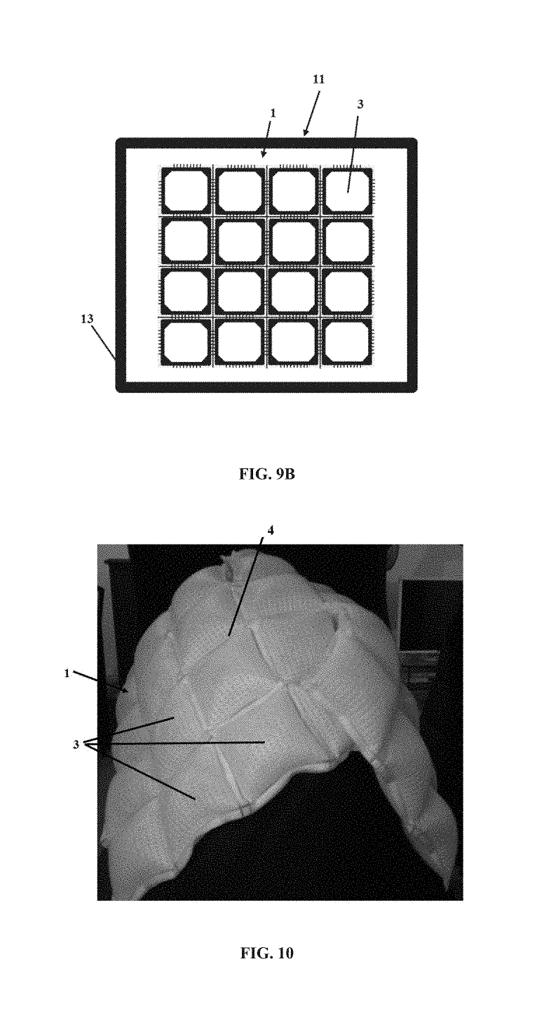

FIGS. 9A and 9B show an article 1 according to the invention, which is included in a single-pocket packaging 11. FIG. 9A shows an article 1 maintained sterile in the packaging 11. In this embodiment, the packaging 11 is closed by any means, in particular via thermowelding of the perimeter of same, which isolates the article 1 from the outside environment in order to retain the sterility of same. In an embodiment, the weld 13 can be peeled. In an embodiment, the packaging 11 comprises at least two adjacent welds that can be peeled. In an embodiment, the activation liquid of the article 1 is a sterile aqueous medium, which is added by means of a prefilled syringe or a prefilled pocket by the intermediary of a means for putting into fluid communication 12; thus, all of the contents of the single-pocket packaging can be retained in conditions of sterility. In the embodiment of FIG. 9B, the packaging comprises a microporous or microperforated sheet (microporosity or microperforation not shown), and all of the perimeter welds 13 of same can be peeled. According to a second embodiment, implemented in a non-sterile framework, the article 1 is removed from the single-pocket packaging 11 so as to allow for the immersion of the article 1 in an activation liquid.

According to a third embodiment, the single-pocket packaging 11 comprises at least one microporous or microperforated portion, in such a way as to be permeable to an aqueous solution in order to allow for the absorption of the activation liquid. In this latter embodiment, the packaging 11 can be plunged directly into an activation liquid, and the article 1 removed from the packaging 11 only after having undergone the thermal transformation of same.

FIG. 10 shows the use of an article 1 according to the invention, for cutaneous application on a knee. Thanks to the flexibility of the article 1, the article covers the knee and provides cold over all of the targeted portions. The article 1 shown in the embodiment of this Figure was partially separated in the length of same on the frangible line or the precut. This separation makes it possible to optimise the covered surface by preventing the formation of a fold and to increase the cold over a portion of the body over which the separated portion of the article is stacked on another portion of this same article.

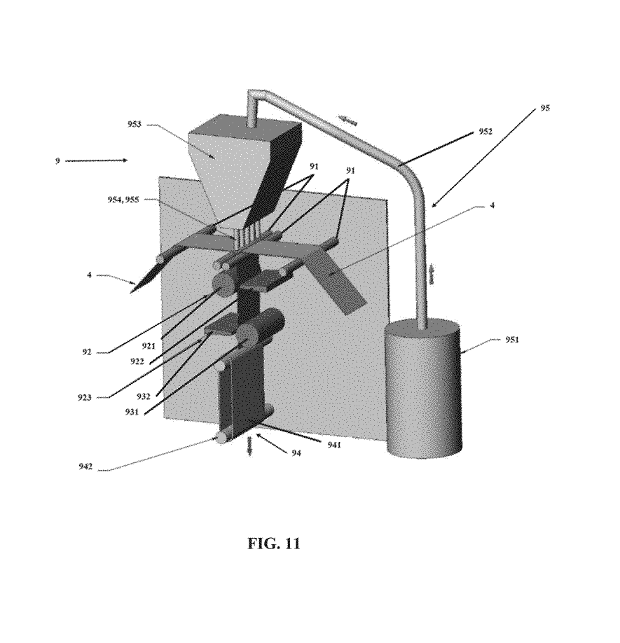

FIG. 11 shows a device for manufacturing 9 an embodiment of the article 1 according to the invention. This device comprises:
two modules for pre-unwinding (not shown) of the sheets 4, woven or non-woven, to supply the sheets 4 without tension,
two units of mechanical parts 91 for guiding and stacking sheets 4, during the fastening operations,
a vertical fastening module 92, making it possible to create several compartments, comprising:
   an ultrasound unit 921 comprising an adjusting unit on rails and pads controlled by a reduction gear, a steel fastening sonotrode, a convertor unit and a 20 KHz ultrasound booster, and
   a fastening drum 922 provided with mechanical parts, fastening thumbwheels for the closing of the pocket and cutting/fastening thumbwheels for the creating of the frangible line or the precut, these thumbwheels can be assembled and can be positioned according to the format of the article to be created and an asynchronous reduction gear,
a horizontal fastening module 93, making it possible to carry out the closing of the pockets comprising the article, comprising:
   an ultrasound unit 931 comprising an adjusting unit on rails and pads controlled by a reduction gear, a steel fastening sonotrode, a convertor unit and a 20 KHz ultrasound booster, and
   a fastening drum 932 provided with mechanical parts, longitudinal fastening bars, and cutting/fastening bars for the creating of the frangible line or of the precut of the pocket in the longitudinal direction and an asynchronous reduction gear,
a vacuum conveyor belt 94 allowing for the motorisation and the transfer of the article produced comprising:
   a set of mechanical parts provided with a perforated sole,
   a perforated conveyor belt 941 allowing for the adherence via vacuum, and
   an asynchronous reduction gear 942,
   a unit for distributing 95 the polyacrylate powder comprising:
      a pneumatic unit for the distribution of the powder from the bag 951 to the intermediate reservoir 953 comprised of a pneumatic unit and a transfer cam 952,
      an intermediate reservoir 953 for the storage of the powder waiting for distribution,
      five mechanical batchers 954 that can be selected allowing for the volumetric metering of the powder to be distributed. Each hatcher is comprised of a set of mechancical parts and of a pneumatic cylinder for the operation of the selection drawer. Each hatcher can be selected in relation with the format of the article to be manufactured, and
      a set of five mechanical corridors 955 that provide the transfer of the powder into the preselected and defined pocket.

1. Article (1) consisting of at least two sheets (4) stacked and welded to define a paving comprising at least three tiles (2), each tile (2) comprising at least three sides and three vertices,
said tiles (2) being deployed in at least two spatial directions defining a plane,
said tiles (2) being adjacent and connected to each other by at least one of the sides of same,
said tiles (2) comprising at least one compartment (3) including at least one material absorbent to an aqueous medium or a filler material,
said tiles (2) being closed and connected to each other by at least one weld,
said weld being produced by ultrasound, heat or any other type of weld known to those skilled in the art,
wherein
the weld consists of a frangible line (8) constituted by a series of weakening welds (6) and junction points (7) or a pre-cut bordered on either side with a mix weld (5),
said mix weld (5) being broadened in the vicinity of each tile (2) such that the corner of the tiles (2) is reinforced by an enlarged weld surface area.

2. Article (1) according to claim 1, wherein at least one of the sheets (4) is formed from a stacking of several thicknesses of material.

3. Article (1) according to claim 1, wherein the weakening weld (6) between two tiles (2) is continuous at the vertex of each tile (2).

4. Article (1) according to claim 1, wherein the weakening weld (6) between two tiles (2) is discontinuous at at least two points per centimetre, in such a way as to generate at least two junction points (7) of mix weld (5).

5. Article (1) according to claim 1, wherein the surface of the compartment (3) occupies 10 to 99% of the surface of the tile (2).

6. Article (1) according to claim 1, wherein the material absorbent to an aqueous medium is the polyacrylate type.

7. Article (1) according to claim 1, wherein at least one of the outer faces of the sheets (4) is coated, entirely or partially, with an adhesive.

8. Article (1) according to claim 1, wherein at least one of the sheets (4) is permeable to an activation liquid, and wherein the material contained in the compartment is a material absorbent to an aqueous medium.

9. Article (1) according to claim 1, wherein said article is in a packaging that comprises at least one microporous or microperforated film permeable to water but a barrier to microbes, bacteria and/or viruses.

10. Article (1) according to claim 1, wherein said article is in a packaging, said packaging comprising:
a first pocket comprising said article (1),
a second pocket, independent of the first pocket and sealed with respect to the first pocket, comprising or able to receive an aqueous medium,
the two pockets being integral via one of the sides of same, and
the two pockets comprising a means of putting into fluid communication from the second pocket to the first pocket.

11. Cooling thermal compress comprising at least one article (1) which, if the material that it contains is a material absorbent to an aqueous medium, was activated by putting into contact with an aqueous medium, and which was cooled, frozen or deep frozen.

12. Roller comprising at least one article (1) according to claim 1 wound on itself.

13. Hat, preferably chemotherapy hat to combat hair loss linked to a chemotherapy treatment, comprising a hairnet, preferably disposable, and at least one article (1) according to claim 1.

14. Method for the continuous manufacture of an article (1) according to claim 1 comprising a continuous flow of at least two non-welded sheets (4) in front of two welding stations: one longitudinal, another transversal; the longitudinal welding station comprising at least three welding zones namely a weakening weld zone (6) bordered on two mix weld zones (5); the transversal welding station comprising at least three welding zones, namely a weakening weld zone (6) bordered on two mix weld zones (5).

15. Device for implementing the method according to claim 14 comprising: a longitudinal welding station comprising at least one means of welding, each means comprising two mix weld washers (5) surrounding a weakening weld washer (6) that can have notches, in such a way as to create a discontinuous weakening weld (6), or without a notch, in such a way as to create a continuous weakening weld (6); and a transversal welding station comprising at least one means of welding, each means comprising two mix weld washers (5) surrounding a weakening weld washer (6) that can have notches, in such a way as to create a discontinuous weakening weld (6), or without a notch, in such a way as to create a continuous weakening weld (6).

16. Article (1) according to claim 1, wherein each one of the two sheets (4) is formed from a stacking of several thicknesses of material.

17. Article (1) according to claim 1, wherein the surface of the compartment (3) occupies 50 to 95% of the surface of the tile (2).

18. Article (1) according to claim 1, wherein the surface of the compartment (3) occupies 70 to 90% of the surface of the tile (2).

19. The article of claim 8, wherein the activation liquid is water or an aqueous medium.

20. A chemotherapy hat to combat hair loss linked to a chemotherapy treatment, comprising a disposable hairnet and at least one article (1) according to claim 1.
